(19) United States
(12) Patent Application Publication
(10) Pub. No.: US 2026/0229326 A1
(43) Pub. Date: Aug. 6, 2026

(54) AI ORCHESTRATION SYSTEM FOR HEALTH DATA ANALYSIS

(71) Applicant: GeoOrchestrationAI Corporation, McLean, VA (US)

(72) Inventors: Ajay Kumar Gupta, Potomac, MD (US); Ramani Peruvemba, McLean, VA (US); Paul A Churchyard, Frostburg, MD (US)

(21) Appl. No.: 19/417,745

(22) Filed: Dec. 12, 2025

Related U.S. Application Data
(60) Provisional application No. 63/753,069, filed on Feb. 3, 2025.

(51) Int. Cl. G16H 10/60; G06F 40/35

(52) U.S. Cl. CPC G16H 10/60; G06F 40/35

(57) ABSTRACT

The present disclosure provides a computer-implemented method for processing health data queries. The method includes receiving a user query through a conversational interface, creating vector embeddings of the user query, comparing the vector embeddings against embeddings of available health metrics stored in a vector database to identify relevant health metrics, extracting geographical parameters from the user query using fine-tuned large language models, retrieving health metric data from a cloud data lake based on the identified relevant health metrics and geographical parameters, filtering the retrieved health metric data based on the geographical parameters, generating summary statistics for the filtered health metric data, and generating multiple output formats including a natural language response and data visualizations.

### FIELD OF INVENTION

The present disclosure relates to artificial intelligence systems for data orchestration and analysis, and more particularly to an AI orchestration system that uses fine-tuned large language models to process natural language queries and generate comprehensive health data insights with geospatial visualizations.

### BACKGROUND

Artificial intelligence systems have become increasingly prevalent in data analysis and decision-making applications across various industries. Traditional AI systems typically focus on specific tasks such as data retrieval, content generation, or pattern recognition, operating within defined parameters and producing outputs based on predetermined algorithms. However, as data volumes continue to grow and become more complex, there is a growing demand for more sophisticated AI systems that can coordinate multiple data processing operations and provide comprehensive insights.

In the healthcare and public health sectors, data analysis presents particular challenges due to the diverse nature of information sources, including demographic data, health metrics, geospatial information, and real-time monitoring data. Healthcare professionals and researchers often work with fragmented datasets that exist in isolation, making it difficult to obtain a complete view of health-related information. This fragmentation can complicate decision-making processes and potentially delay responses to public health concerns.

Generative AI models have shown promise in understanding and responding to natural language queries in a conversational manner. These models can generate coherent responses and provide explanations that feel natural to users. However, generative AI systems operating independently may lack contextual accuracy and the ability to reliably access and integrate external data sources. Additionally, such systems may be susceptible to generating plausible but factually incorrect information, which can undermine trust and lead to poor decision-making, particularly in applications where accuracy is paramount.

Current data analysis systems often require users to possess technical expertise in data science, programming, or specialized software tools. This technical barrier can limit access to valuable insights for decision-makers who may not have the time or background to interpret raw data or navigate complex analytical interfaces. There is a growing need for systems that can bridge the gap between complex data analysis capabilities and user-friendly interfaces that enable natural language interaction.

The integration of geospatial data with health metrics presents additional complexity, as it requires coordination between different data formats, coordinate systems, and analytical approaches. Traditional systems may handle geospatial visualization or health data analysis separately, but combining these capabilities in a seamless manner while maintaining accuracy and relevance to user queries remains challenging.

Therefore, improved systems and methods that can orchestrate multiple data sources, provide accurate and contextually relevant responses to natural language queries, and present information in accessible formats would be beneficial for advancing data-driven decision-making in healthcare and related fields.

### SUMMARY

This summary is provided to introduce a selection of concepts in a simplified form that are further described below in the detailed description. This summary is not intended to identify key features or essential features of the claimed subject matter, nor is it intended to be used as an aid in determining the scope of the claimed subject matter.

According to an aspect of the present disclosure, a computer-implemented method for processing health data queries is provided. The method comprises receiving a user query through a conversational interface. The method comprises creating vector embeddings of the user query. The method comprises comparing the vector embeddings against embeddings of available health metrics stored in a vector database to identify relevant health metrics. The method comprises extracting geographical parameters from the user query using fine-tuned large language models. The method comprises retrieving health metric data from a cloud data lake based on the identified relevant health metrics and geographical parameters. The method comprises filtering the retrieved health metric data based on the geographical parameters. The method comprises generating summary statistics for the filtered health metric data. The method comprises generating multiple output formats including a natural language response and data visualizations.

According to other aspects of the present disclosure, the method may include one or more of the following features. The fine-tuned large language models may comprise specialized functions for selecting metric extent, selecting relevant metrics, selecting geometry levels, checking additional parameters, selecting relevant geometries, selecting administrative filters, selecting administrative filter values, and selecting geometry names. The method may comprise initiating cloud-based data processing pipelines when health metric data is not immediately available. The method may comprise retrieving geospatial data layers corresponding to the identified health metrics. The method may comprise performing intersection overlay geospatial operations when the geospatial data comprises polygon geometries. The data visualizations may comprise maps, tables, pie charts, and bar charts. The method may comprise storing chat history in a database and updating user queries based on previous interactions. The natural language response may be generated using retrieval augmented generation from curated authoritative sources.

According to another aspect of the present disclosure, an AI orchestration system for health data analysis is provided. The system comprises a conversational interface configured to receive user queries. The system comprises a vector database storing embeddings of available health metrics. The system comprises fine-tuned large language models configured to extract parameters from user queries. The system comprises a cloud data lake storing health metric datasets and geospatial data. The system comprises a data orchestrator configured to retrieve and filter data based on extracted parameters. The system comprises a visualization engine configured to generate multiple output formats including natural language responses and data visualizations.

According to other aspects of the present disclosure, the system may include one or more of the following features. The fine-tuned large language models may comprise picker functions that select relevant options from available datasets based on user queries. The system may comprise cloud data pipeline components including an event bus and lambda functions for processing data requests. The system may comprise a web deployment infrastructure including containerized applications and domain management services. The data orchestrator may be configured to perform geospatial operations including intersection overlays and administrative boundary filtering. The visualization engine may generate maps, tables, charts, and downloadable data exports. The system may comprise authentication and access control mechanisms for user management.

The foregoing general description of the illustrative embodiments and the following detailed description thereof are merely exemplary aspects of the teachings of this disclosure and are not restrictive.

### BRIEF DESCRIPTION OF FIGURES

Non-limiting and non-exhaustive examples are described with reference to the following figures.

FIG. 1 illustrates a flowchart of a user query processing method, according to aspects of the present disclosure.

FIG. 2 depicts a flowchart of a metric retrieval and processing method, according to aspects of the present disclosure.

FIG. 3 illustrates a flowchart of a data filtering method, according to aspects of the present disclosure.

FIG. 4 depicts a flowchart of a query response generation method, according to aspects of the present disclosure.

FIG. 5 illustrates a block diagram of an AWS cloud infrastructure with interconnected components, according to aspects of the present disclosure.

FIG. 6 depicts a block diagram of an AI orchestration system with LLM function categories, according to aspects of the present disclosure.

### DETAILED DESCRIPTION

The following description sets forth exemplary aspects of the present disclosure. It should be recognized, however, that such description is not intended as a limitation on the scope of the present disclosure. Rather, the description also encompasses combinations and modifications to those exemplary aspects described herein.

The present disclosure relates to an artificial intelligence orchestration system designed for health data analysis and decision support. The system may process natural language queries from users and generate comprehensive health data insights accompanied by geospatial visualizations. In some cases, the system may coordinate multiple data processing operations to transform complex health datasets into actionable information that can be readily understood and utilized by decision-makers across various sectors including public health, emergency management, and healthcare resource allocation.

The orchestration system may function as a central coordination hub that harmonizes disparate data elements from multiple sources to deliver tailored insights. In some cases, the system may integrate generative artificial intelligence capabilities with intelligent data retrieval and processing mechanisms to address challenges commonly associated with fragmented health data. The system may enable users to submit queries through conversational interfaces without requiring technical expertise in data analysis or programming skills. Through natural language comprehension, the system may parse user requests, identify relevant health metrics, extract appropriate parameters, and orchestrate the retrieval and processing of data from authoritative sources.

The system may address limitations commonly found in traditional generative AI approaches by incorporating robust data orchestration processes that draw from curated, authoritative datasets. In some cases, the system may minimize risks associated with generating inaccurate or misleading information by grounding responses in verified data sources rather than relying solely on generative capabilities. The orchestration approach may enable the system to provide reliable, contextually relevant information for decision-making in areas where accuracy may be particularly valuable, such as healthcare and emergency management scenarios.

The orchestration system may support multiple output formats to accommodate diverse user needs and preferences. In some cases, the system may generate natural language responses, geospatial maps, data tables, statistical reports, and various chart visualizations based on the same underlying query. The multi-modal output capability may enhance accessibility to complex health risk insights and facilitate information sharing among stakeholders with varying technical backgrounds. The system may also incorporate real-time data processing capabilities to ensure that users receive current and relevant information for time-sensitive decision-making scenarios.

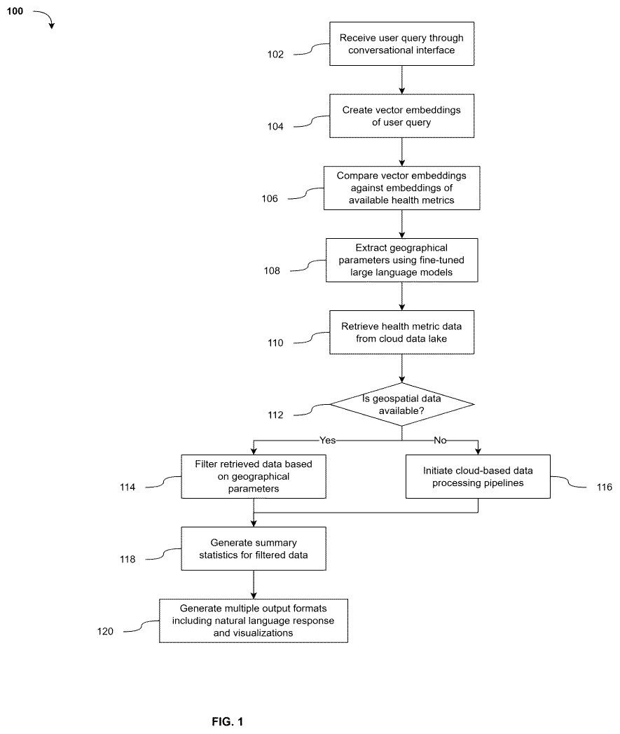

Referring to FIG. 1, a user query processing method 100 may be implemented to handle natural language queries submitted through conversational interfaces and transform such queries into comprehensive health data insights. The method 100 may begin at a step 102 where the system receives a user query through a conversational interface. In some cases, the conversational interface may provide users with a text input mechanism that allows submission of queries without requiring technical expertise in data analysis or programming skills. The method 100 may incorporate chat history integration capabilities that utilize DynamoDB tables for real-time tracking of user interactions without race condition concerns. The chat history integration may enable the system to maintain context across multiple query sessions and provide more accurate responses based on previous user interactions.

Following the query reception at step 102, the method 100 may proceed to a step 104 where vector embeddings of the user query are created. The vector embedding creation process may transform the natural language query into numerical representations that can be processed by machine learning algorithms and compared against existing data structures. In some cases, the vector embeddings may capture semantic meaning and contextual relationships within the user query to enable more accurate matching with available health metrics and datasets. The embedding process may utilize advanced natural language processing techniques to ensure that the numerical representations preserve the intent and meaning of the original query text.

The method 100 may continue to a step 106 where the vector embeddings are compared against embeddings of available health metrics. This comparison process may involve semantic similarity matching algorithms that identify the most relevant health metrics based on the content and context of the user query. In some cases, the system may maintain a comprehensive database of health metric embeddings that encompasses various categories of health data including social determinants of health, disease prevalence indicators, healthcare access metrics, and environmental health factors. The comparison process may utilize maximal marginal relevance techniques to ensure that the selected metrics provide diverse and comprehensive coverage of the query topic while avoiding redundant or overly similar data sources.

As further shown in FIG. 1, the method 100 may advance to a step 108 where geographical parameters are extracted using fine-tuned large language models. The geographical parameter extraction process may identify specific locations, administrative boundaries, or spatial scales mentioned in the user query to ensure that the retrieved data corresponds to the appropriate geographic scope. In some cases, the fine-tuned large language models may be trained to recognize various forms of geographic references including state names, county designations, ZIP code areas, census tract identifiers, and other administrative boundary specifications. The parameter extraction may also determine the appropriate spatial resolution for the analysis based on the context and requirements of the user query.

The method 100 may proceed to a step 110 where health metric data is retrieved from a cloud data lake based on the identified metrics and geographical parameters. The data retrieval process may access structured datasets stored in distributed cloud storage systems and filter the data according to the specified geographic boundaries and temporal parameters. In some cases, the cloud data lake may contain multiple years of health data organized by geographic levels and metric categories to support diverse analytical requirements. The retrieval process may incorporate data validation mechanisms to ensure that the accessed datasets meet quality standards and contain the information needed to address the user query.

With continued reference to FIG. 1, the method 100 may include a decision step 112 that determines whether geospatial data is available for the selected metrics and geographic parameters. The availability determination may involve checking the cloud data lake for corresponding geospatial layers, boundary files, or coordinate information that can be used to create map visualizations and perform spatial analyses. In some cases, the geospatial data availability may depend on the specific geographic scale requested and the nature of the health metrics being analyzed. The decision logic at step 112 may direct the processing flow to different pathways based on the availability of geospatial components.

When geospatial data is available, the method 100 may continue to a step 114 where retrieved data is filtered based on geographical parameters. The filtering process may apply spatial constraints to limit the dataset to the specific geographic areas of interest and ensure that the analysis focuses on the relevant geographic scope. In some cases, the filtering may involve intersection operations between health metric data and geographic boundary layers to identify data points that fall within the specified administrative areas or spatial extents. The geographical filtering may also incorporate buffer zones or proximity analyses when appropriate for the type of health metric being examined.

Alternatively, when geospatial data is not available as determined at step 112, the method 100 may branch to a step 116 where cloud-based data processing pipelines are initiated. The pipeline initiation may trigger automated data processing workflows that generate the required geospatial components or perform alternative analyses that do not depend on spatial visualization capabilities. In some cases, the cloud-based pipelines may include data synthesis operations, statistical modeling procedures, or data transformation processes that create the information needed to respond to the user query. The pipeline initiation may also include notification mechanisms that inform users about processing timeframes and expected completion times for complex analytical operations.

Following either the data filtering at step 114 or the pipeline initiation at step 116, the method 100 may advance to a step 118 where summary statistics are generated for filtered data. The summary statistics generation may include calculations of descriptive measures such as means, medians, standard deviations, quartiles, and other statistical indicators that characterize the distribution and central tendencies of the health metric data. In some cases, the statistical analysis may also include identification of extreme values, outliers, or notable patterns within the dataset that may be relevant to the user query. The summary statistics may provide quantitative foundations for the natural language responses and support evidence-based interpretations of the health data patterns.

The method 100 may conclude at a step 120 where multiple output formats are generated including natural language response and visualizations. The output generation process may create diverse presentation formats to accommodate different user preferences and communication needs. In some cases, the multiple output formats may include geospatial maps that display the health metric data in geographic context, data tables that present detailed numerical information, statistical reports that summarize key findings, and various chart types such as bar charts and pie charts that illustrate data patterns and relationships. The natural language response generation may utilize fine-tuned language models to create coherent explanations of the analysis results that are accessible to users without technical backgrounds in data analysis or statistics.

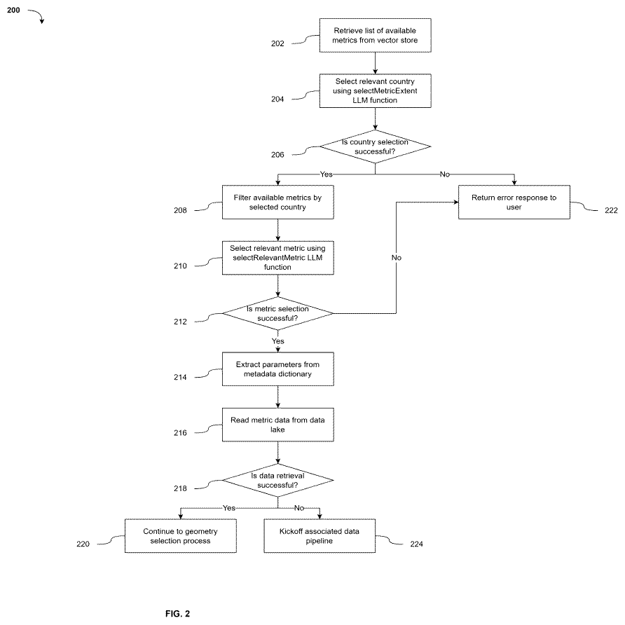

Referring to FIG. 2, a metric retrieval and processing method 200 may be implemented to identify and retrieve health metrics from distributed data sources based on natural language query analysis. The method 200 may begin at a step 202 where the system retrieves a list of available metrics from a vector store. In some cases, the vector store may contain embeddings for over 600 available metrics across multiple countries, with each metric represented as numerical vectors that capture semantic relationships and contextual information. The retrieval process may access a ChromaDB vector store hosted on cloud infrastructure to obtain comprehensive listings of health indicators, demographic data, environmental factors, and healthcare access measurements that can be utilized for query processing. The available metrics may encompass diverse categories including social determinants of health indices, disease prevalence statistics, healthcare facility locations, and population demographic characteristics organized by geographic administrative levels.

The method 200 may proceed to a step 204 where a relevant country is selected using a selectMetricExtent LLM function. The selectMetricExtent LLM function may analyze the user query to identify geographical references and match such references against available country options within the metric database. In some cases, the LLM function may be trained to recognize various forms of country names, abbreviations, and geographic identifiers to ensure accurate country selection based on query content. The function may utilize natural language comprehension capabilities to parse geographic context from user queries and return the most appropriate country designation for subsequent data filtering operations. The selectMetricExtent function may also handle cases where multiple countries are referenced or where geographic scope may be ambiguous within the query text.

Following the country selection at step 204, the method 200 may advance to a decision step 206 that determines whether the country selection process completed successfully. The decision logic at step 206 may evaluate whether the selectMetricExtent LLM function returned a valid country identifier or produced a null response indicating that no suitable country match was found within the available options. In some cases, the decision step may also validate that the selected country corresponds to geographic regions where health metric data is available within the system databases. The validation process may check data availability and coverage to ensure that subsequent processing steps can access appropriate datasets for the identified geographic scope.

When the country selection is successful as determined at step 206, the method 200 may continue to a step 208 where available metrics are filtered by the selected country. The filtering process may reduce the comprehensive metric database to a subset that contains only those health indicators and datasets that are available for the identified country or geographic region. In some cases, the filtering operation may significantly reduce the number of metrics under consideration to create a more manageable dataset for subsequent processing steps. The country-based filtering may also incorporate temporal considerations to ensure that the filtered metrics include data for appropriate time periods and maintain consistency across different geographic administrative levels within the selected country.

As further shown in FIG. 2, the method 200 may advance to a step 210 where a relevant metric is selected using a selectRelevantMetric LLM function. The selectRelevantMetric LLM function may analyze the filtered metric options against the user query content to identify the most appropriate health indicator or dataset for addressing the query requirements. In some cases, the function may utilize semantic similarity matching algorithms to compare query embeddings with metric embeddings and determine the closest conceptual matches. The selectRelevantMetric function may also consider contextual factors such as the type of analysis requested, the geographic scale specified, and the temporal scope mentioned in the query to ensure that the selected metric aligns with user intentions and analytical requirements.

The method 200 may proceed to a decision step 212 that determines whether the metric selection process completed successfully. The decision logic at step 212 may evaluate whether the selectRelevantMetric LLM function identified a suitable health metric from the filtered options or returned a null response indicating that no appropriate match was found. In some cases, the decision step may also validate that the selected metric contains sufficient data coverage for the specified geographic and temporal parameters. The validation process may check data completeness, quality indicators, and availability across the requested administrative levels to ensure that the selected metric can support the intended analysis operations.

When the metric selection is not successful as determined at step 212, the method 200 may branch to a step 222 where an error response is returned to the user. The error response generation may create informative messages that explain the nature of the selection failure and provide guidance for query modification or alternative approaches. In some cases, the error response may include suggestions for available metrics that are conceptually related to the original query or recommendations for adjusting geographic or temporal parameters to access suitable datasets. The error handling mechanism may also provide users with listings of available metric categories and geographic coverage areas to facilitate successful query reformulation.

With continued reference to FIG. 2, when the metric selection is successful as determined at step 212, the method 200 may continue to a step 214 where parameters are extracted from a metadata dictionary. The parameter extraction process may utilize regular expression parsing techniques to identify specific parameters needed for reading correct data files that are organized by year, country, geographic level, and other categorical factors. In some cases, the metadata dictionary may contain comprehensive information about data file locations, naming conventions, temporal coverage, geographic scope, and additional parameters that govern data access and retrieval operations. The parameter extraction may also identify data quality indicators, update frequencies, and source attribution information that may be relevant for subsequent processing and response generation activities.

The method 200 may advance to a step 216 where metric data is read from a data lake based on the extracted parameters. The data reading process may access distributed cloud storage systems to retrieve the specific datasets that correspond to the selected metric and geographic parameters. In some cases, the data lake may contain multiple file formats and organizational structures that require parameter-specific access protocols to ensure accurate data retrieval. The reading operation may also incorporate data validation mechanisms to verify file integrity, format consistency, and content completeness before proceeding with subsequent processing steps. The data lake access may utilize cloud-based storage services that provide scalable and reliable data retrieval capabilities for large-scale health datasets.

Following the data reading attempt at step 216, the method 200 may proceed to a decision step 218 that determines whether the data retrieval process completed successfully. The decision logic at step 218 may evaluate whether the data files were successfully accessed, read, and loaded into memory for subsequent processing operations. In some cases, the decision step may also validate data format consistency, completeness of required fields, and alignment with expected data structures based on the metadata specifications. The validation process may check for missing values, data type consistency, and geographic identifier accuracy to ensure that the retrieved data can support the intended analytical operations and visualization requirements.

When data retrieval is successful as determined at step 218, the method 200 may continue to a step 220 where the process advances to geometry selection operations. The transition to geometry selection may enable the system to identify and retrieve corresponding geospatial data layers that can be used for map visualization and spatial analysis operations. In some cases, the geometry selection process may access vector boundary files, coordinate systems, and spatial reference information that align with the retrieved health metric data. The progression to geometry selection may also trigger additional parameter extraction and validation processes to ensure compatibility between metric datasets and geospatial components.

Alternatively, when data retrieval is not successful as determined at step 218, the method 200 may branch to a step 224 where associated data pipelines are initiated. The pipeline initiation process may trigger automated data processing workflows through Event Bridge events that generate the required datasets when such datasets are not currently available in the data lake. In some cases, the pipeline initiation may include 20-minute processing time estimates that inform users about expected completion timeframes for complex analytical operations. The automated pipelines may perform data synthesis, statistical modeling, geographic aggregation, or other computational processes that create the health metric datasets needed to address the user query. The pipeline initiation may also include notification mechanisms that update users about processing status and provide options for query resubmission after pipeline completion.

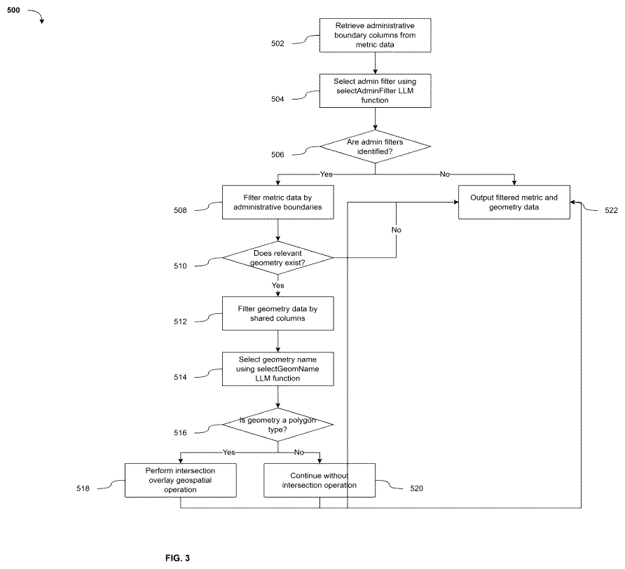

Referring to FIG. 3, a data filtering method 500 may be implemented to process and spatially constrain health metric data based on administrative boundaries and geospatial parameters extracted from user queries. The method 500 may begin at a step 502 where administrative boundary columns are retrieved from metric data that was previously obtained through the metric retrieval and processing operations. In some cases, the administrative boundary columns may include various geographic identifiers such as state names, county designations, ZIP code areas, census tract numbers, and other hierarchical administrative divisions that organize health data according to governmental and statistical boundaries. The retrieval process may access column headers and metadata information to identify which administrative levels are available within the specific health metric dataset being processed. The administrative boundary identification may also determine the geographic scope and resolution capabilities of the dataset to inform subsequent filtering operations.

The method 500 may proceed to a step 504 where an admin filter is selected using a selectAdminFilter LLM function. The selectAdminFilter LLM function may analyze the user query content to identify which administrative boundary levels are referenced or implied within the query text and match such references against the available administrative columns within the health metric dataset. In some cases, the LLM function may be trained to recognize various forms of geographic references including formal administrative names, common abbreviations, and colloquial geographic identifiers that users may employ when specifying areas of interest. The selectAdminFilter function may utilize natural language comprehension capabilities to parse geographic context and determine which administrative levels provide the most appropriate filtering scope for the user query. The function may also handle cases where multiple administrative levels are mentioned or where geographic scope may be hierarchically nested within the query specifications.

Following the admin filter selection at step 504, the method 500 may advance to a decision step 506 that determines whether admin filters are identified successfully. The decision logic at step 506 may evaluate whether the selectAdminFilter LLM function returned valid administrative boundary column identifiers or produced a null response indicating that no suitable administrative filters were found within the available dataset structure. In some cases, the decision step may also validate that the identified administrative columns contain sufficient data coverage and geographic specificity to support meaningful filtering operations. The validation process may check for data completeness across the identified administrative levels and ensure that the selected filters align with the geographic scope and analytical requirements specified in the user query.

When admin filters are identified successfully as determined at step 506, the method 500 may continue to a step 508 where metric data is filtered by administrative boundaries. The filtering process may apply geographic constraints to limit the health metric dataset to specific administrative areas that correspond to the user query requirements and ensure that subsequent analyses focus on the relevant geographic scope. In some cases, the administrative boundary filtering may involve multiple hierarchical levels simultaneously, such as filtering by state and then by county within the selected state to create nested geographic constraints. The filtering operation may also incorporate data validation mechanisms to ensure that the filtered dataset maintains referential integrity and contains sufficient data points to support statistical analyses and visualization operations. The administrative filtering may utilize indexing and query optimization techniques to efficiently process large health datasets while maintaining data accuracy and completeness.

As further shown in FIG. 3, the method 500 may advance to a decision step 510 that determines whether relevant geometry exists for the filtered health metric data. The decision logic at step 510 may evaluate whether corresponding geospatial data layers, boundary files, or coordinate information are available to support map visualization and spatial analysis operations for the administratively filtered dataset. In some cases, the geometry availability determination may depend on the specific administrative levels selected and the geographic scale of the analysis, as different administrative boundaries may have varying levels of geospatial data support within the system databases. The decision step may also check for compatibility between the filtered metric data and available geometry datasets to ensure that spatial joins and overlay operations can be performed successfully.

When relevant geometry does not exist as determined at step 510, the method 500 may branch to a step 522 where filtered metric and geometry data is output for subsequent processing operations. The output generation may create data structures that contain the administratively filtered health metric information along with any available geospatial components that can support visualization and analysis activities. In some cases, the output may include metadata information about the filtering operations performed and the geographic scope of the resulting dataset to inform subsequent processing steps and user response generation. The output formatting may also prepare the filtered data for integration with visualization tools and statistical analysis functions that generate the final query responses.

Alternatively, when relevant geometry exists as determined at step 510, the method 500 may proceed to a step 512 where geometry data is filtered by shared columns between the health metric dataset and the geospatial data layers. The shared column filtering process may identify common geographic identifiers such as administrative boundary codes, geographic names, or coordinate reference systems that enable spatial joins between the metric data and geometry components. In some cases, the filtering operation may involve multiple shared columns to ensure accurate spatial alignment and prevent mismatched geographic associations between health data and boundary representations. The geometry filtering may also incorporate data validation mechanisms to verify that the shared columns contain consistent formatting, naming conventions, and geographic coverage across both the metric and geometry datasets.

With continued reference to FIG. 3, the method 500 may continue to a step 514 where a geometry name is selected using a selectGeomName LLM function. The selectGeomName LLM function may analyze the user query content to identify specific geographic features, boundary names, or spatial entities that are explicitly mentioned within the query text and match such references against available geometry names within the filtered geospatial dataset. In some cases, the LLM function may be trained to recognize various forms of geographic nomenclature including official boundary names, alternative geographic designations, and contextual spatial references that users may employ when specifying areas of interest. The selectGeomName function may utilize semantic similarity matching algorithms to compare query content with geometry name embeddings and identify the most appropriate spatial features for the analysis. The function may also handle cases where multiple geometry names are referenced or where spatial scope may be ambiguous within the query specifications.

The method 500 may advance to a decision step 516 that determines whether the selected geometry is a polygon type rather than point or line geometries. The geometry type determination may evaluate the spatial data structure and geometric properties of the selected geospatial features to identify whether they represent area-based boundaries that can support intersection overlay operations with the health metric data. In some cases, polygon geometries may include administrative boundaries, census areas, health service regions, or other area-based geographic features that define spatial extents for health data analysis. The decision logic at step 516 may also validate that the polygon geometries contain sufficient spatial detail and coordinate accuracy to support precise intersection calculations with the health metric data points or geographic identifiers.

When the geometry is determined to be a polygon type as evaluated at step 516, the method 500 may continue to a step 518 where an intersection overlay geospatial operation is performed to spatially filter the health metric data. The intersection overlay operation may utilize computational geometry algorithms to identify health data points, administrative areas, or geographic identifiers that fall within the spatial boundaries defined by the selected polygon geometries. In some cases, the intersection operation may involve complex spatial calculations that account for coordinate system transformations, boundary precision, and edge case handling to ensure accurate spatial filtering results. The geospatial operation may also incorporate buffer zones, proximity analyses, or other spatial relationship calculations when appropriate for the type of health metric being examined and the spatial scale of the analysis. The intersection overlay may utilize specialized geospatial processing libraries and algorithms that provide efficient and accurate spatial filtering capabilities for large-scale health datasets.

Alternatively, when the geometry is not a polygon type as determined at step 516, the method 500 may proceed to a step 520 where the process continues without intersection operation. The continuation without intersection may indicate that the selected geometry consists of point or line features that do not define area-based spatial extents suitable for overlay operations with the health metric data. In some cases, the processing may continue with alternative spatial analysis approaches such as proximity calculations, buffer analyses, or distance-based filtering operations that are more appropriate for point or line geometries. The alternative processing pathway may also maintain the geometry information for visualization purposes while applying different spatial relationship calculations that align with the geometric properties of the selected spatial features.

From either step 518 or step 520, the method 500 may conclude at step 522 where filtered metric and geometry data is output for subsequent processing and response generation operations. The output generation may create comprehensive data structures that contain both the spatially filtered health metric information and the corresponding geospatial components that support map visualization and spatial analysis activities. In some cases, the output may include detailed metadata about the filtering operations performed, the spatial relationships identified, and the geographic scope of the resulting dataset to inform subsequent processing steps and ensure accurate interpretation of the filtered results. The output formatting may also prepare the filtered data for integration with statistical analysis functions, visualization tools, and natural language response generation systems that create the final user query responses. The filtered metric and geometry data may maintain spatial referential integrity and include coordinate system information that enables accurate map rendering and geospatial visualization capabilities.

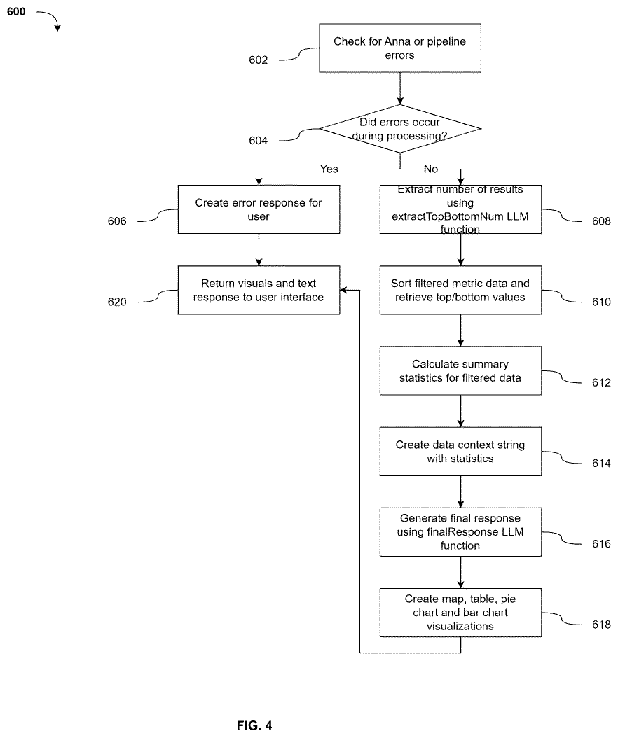

Referring to FIG. 4, a query response generation method 600 may be implemented to process filtered health metric data and generate comprehensive user responses that include error handling, statistical analysis, and multiple visualization formats. The method 600 may begin at a step 602 where the system checks for errors that may have occurred during previous processing operations including metric selection, data retrieval, geographical filtering, or geospatial processing activities. In some cases, the error checking process may evaluate the status of multiple processing components to identify failures in data access, parameter extraction, administrative boundary filtering, or geometry selection operations that could affect the quality or completeness of the query response. The error detection mechanisms may also assess data validation results, file integrity checks, and processing pipeline status indicators to determine whether sufficient information is available to generate meaningful responses to user queries. The comprehensive error checking may enable the system to provide appropriate feedback to users when processing limitations or data availability constraints prevent complete query fulfillment.

Following the error checking at step 602, the method 600 may advance to a decision step 604 that determines whether errors occurred during the preceding processing operations. The decision logic at step 604 may evaluate error flags, exception conditions, and validation results from multiple processing stages to determine the overall success status of the query processing workflow. In some cases, the decision step may distinguish between different types of errors such as data availability issues, parameter extraction failures, or geospatial processing limitations that may require different response strategies. The error evaluation process may also consider the severity and impact of detected errors to determine whether partial responses can be generated or whether complete error handling procedures should be initiated. The decision logic may incorporate threshold-based assessments that allow for minor processing issues while identifying significant errors that prevent meaningful query responses.

When errors are detected as determined at step 604, the method 600 may proceed to a step 606 where an error response is created for the user. The error response generation process may create informative messages that explain the nature of the processing failures and provide guidance for query modification or alternative analytical approaches. In some cases, the error response may include specific information about data availability limitations, geographic coverage constraints, or temporal scope restrictions that prevented successful query processing. The error message creation may also provide users with suggestions for adjusting query parameters, selecting alternative metrics, or modifying geographic specifications to access available datasets and analytical capabilities. The error response formatting may ensure that technical processing details are translated into user-friendly explanations that enable effective query refinement and resubmission.

Alternatively, when no errors are detected as determined at step 604, the method 600 may continue to a step 608 where the number of results is extracted using an extractTopBottomNum LLM function. The extractTopBottomNum LLM function may analyze the user query content to identify numerical specifications that indicate how many results the user wishes to receive in the response, such as requests for the top ten areas with highest risk values or the five counties with lowest healthcare access scores. In some cases, the LLM function may be trained to recognize various forms of numerical requests including explicit numbers, ordinal references, and contextual quantity indicators that users may employ when specifying result scope preferences. The function may utilize natural language comprehension capabilities to parse numerical context from query text and return appropriate result count specifications for subsequent data processing operations. The extractTopBottomNum function may also handle cases where no specific result quantities are mentioned by applying default values that provide comprehensive yet manageable response sizes.

As further shown in FIG. 4, the method 600 may proceed to a step 610 where filtered metric data is sorted and top/bottom values are retrieved based on the extracted result count specifications. The sorting process may organize the health metric data in ascending or descending order according to the metric values to identify the highest and lowest performing geographic areas, administrative boundaries, or data points within the filtered dataset. In some cases, the sorting operation may handle multiple data types including numerical health indicators, categorical risk classifications, and ordinal rating systems that require different comparison and ranking algorithms. The top and bottom value retrieval may extract the specified number of results from both ends of the sorted distribution to provide users with comprehensive perspectives on the range and variation within the health metric data. The value extraction process may also maintain geographic identifiers, administrative boundary information, and associated metadata that enable accurate interpretation and presentation of the selected results.

The method 600 may advance to a step 612 where summary statistics are calculated for the filtered data to provide quantitative characterizations of the health metric distributions and central tendencies. The summary statistics calculation process may generate comprehensive statistical measures including count, mean, standard deviation, minimum, 25th percentile, 50th percentile, 75th percentile, maximum, and sum values that describe the mathematical properties of the filtered health metric dataset. In some cases, the statistical calculations may also include additional measures such as variance, skewness, kurtosis, and confidence intervals that provide deeper insights into the distributional characteristics and statistical significance of the health data patterns. The summary statistics generation may incorporate data validation mechanisms to handle missing values, outliers, and data quality issues that could affect the accuracy and reliability of the calculated measures. The statistical analysis may also account for different data types and measurement scales within the health metrics to ensure that appropriate statistical methods are applied for each type of health indicator being analyzed.

With continued reference to FIG. 4, the method 600 may continue to a step 614 where a data context string is created with the statistics and top/bottom values to provide structured information for subsequent natural language response generation. The data context string creation process may combine the calculated summary statistics, extracted top and bottom values, geographic identifiers, and relevant metadata into formatted text structures that can be processed by language generation models. In some cases, the context string may also incorporate information about administrative boundary filters applied, geometry selection results, and data processing operations performed to provide comprehensive background information for response generation. The context string formatting may organize the statistical and geographic information in logical sequences that facilitate coherent natural language explanations and ensure that all relevant analytical results are included in the final user responses. The data context preparation may also include integration of retrieval augmented generation content from document vector stores that provide additional contextual information about the health metrics, geographic areas, or analytical methods being utilized.

The method 600 may proceed to a step 616 where a final response is generated using a finalResponse LLM function that processes the data context string and user query to create coherent natural language explanations of the analytical results. The finalResponse LLM function may utilize advanced natural language generation capabilities to transform statistical information, geographic data, and analytical results into accessible explanations that can be understood by users without technical backgrounds in data analysis or statistics. In some cases, the function may be trained to generate responses that maintain scientific accuracy while using clear and engaging language that facilitates effective communication of health data insights. The final response generation may also incorporate contextual information about data sources, analytical methods, and geographic scope to provide users with comprehensive understanding of the analysis basis and limitations. The finalResponse function may handle various response formats including detailed explanations, summary statements, and action-oriented recommendations that align with different user needs and decision-making contexts.

Following the final response generation at step 616, the method 600 may advance to a step 618 where map, table, pie chart and bar chart visualizations are created to provide multiple presentation formats that accommodate diverse user preferences and communication requirements. The visualization generation process may create geospatial maps that display the health metric data in geographic context using coordinate systems, boundary representations, and symbology that accurately represent the spatial distribution and patterns within the filtered dataset. In some cases, the visualization creation may also generate data tables that present detailed numerical information in structured formats that enable precise examination of individual data points, administrative areas, and statistical measures. The pie chart generation may create circular visualizations that illustrate proportional relationships and categorical distributions within the health metric data, while bar chart creation may produce comparative visualizations that highlight differences and rankings among geographic areas or administrative boundaries. The multi-modal output generation may ensure that users receive comprehensive information packages that support different analytical needs and presentation contexts.

The method 600 may conclude at a step 620 where visuals and text response are returned to the user interface along with data export functionality that enables users to access and download query results and visualizations. The response return process may integrate the generated natural language explanations, statistical summaries, and multiple visualization formats into cohesive user interface presentations that provide immediate access to analytical results and supporting information. In some cases, the response delivery may also include export functionality to temporary S3 buckets that provide download links for users to access detailed datasets, high-resolution visualizations, and comprehensive reports that extend beyond the immediate interface display capabilities. The data export functionality may generate multiple file formats including spreadsheet documents, image files, geographic data formats, and structured reports that accommodate different user workflow requirements and downstream analytical applications. The response return process may also update chat history records and maintain session information that enables continued interaction and follow-up query processing based on the current analytical results and user engagement patterns.

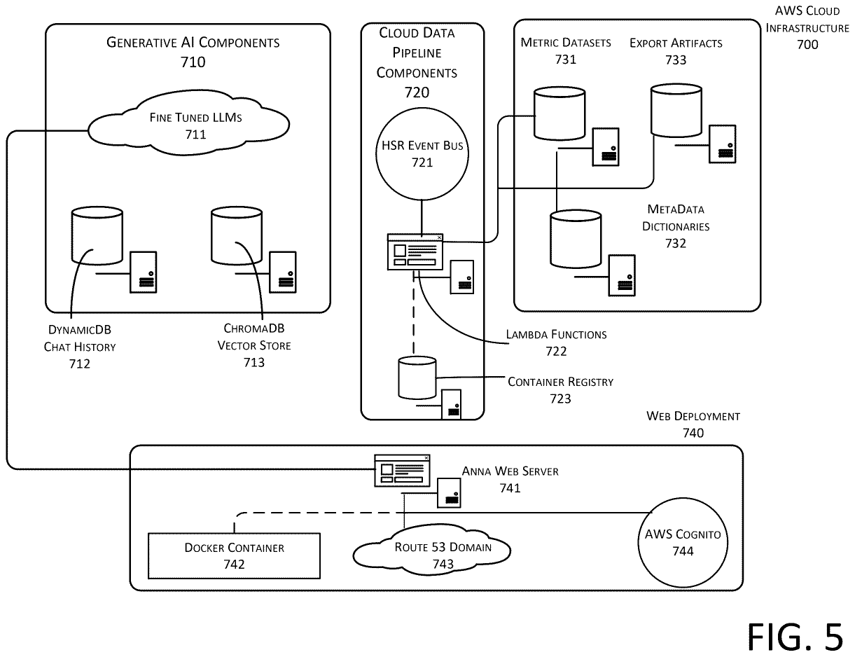

Referring to FIG. 5, an AWS Cloud Infrastructure 700 may be implemented to support the orchestration system through distributed cloud computing services that provide scalable data processing, storage, and web deployment capabilities. The AWS Cloud Infrastructure 700 may comprise four main component groups that work together to enable comprehensive health data analysis and query processing operations. In some cases, the infrastructure architecture may utilize cloud-based services to provide reliable, scalable, and secure computing resources that can handle large-scale health datasets and support multiple concurrent user sessions. The distributed architecture may enable the system to process complex analytical operations while maintaining responsive user interfaces and ensuring data security across multiple processing stages. The AWS Cloud Infrastructure 700 may also incorporate redundancy and fault tolerance mechanisms that ensure continuous system availability and data integrity during high-demand periods or component maintenance activities.

The first component group within the AWS Cloud Infrastructure 700 may include Generative AI Components 710 that provide natural language processing and machine learning capabilities for query interpretation and response generation. The Generative AI Components 710 may encompass Fine-Tuned LLMs 711 that utilize specialized language models trained for specific tasks within the orchestration workflow, such as metric selection, geographic parameter extraction, and natural language response generation. In some cases, the Fine-Tuned LLMs 711 may be implemented using OpenAI API with GPT-4 Omni foundational models that provide advanced natural language comprehension and generation capabilities for processing user queries and creating coherent responses. The Fine-Tuned LLMs 711 may also be implemented using AWS Bedrock with AWS NOVA foundational models that offer cloud-native language processing services integrated with other AWS infrastructure components. The Generative AI Components 710 may also include a DynamoDB Chat History 712 that stores user interaction records and conversation context information using NoSQL database services that provide fast access and real-time tracking capabilities without race condition concerns.

As further shown in FIG. 5, the Generative AI Components 710 may also incorporate a ChromaDB Vector Store 713 that maintains embeddings for available metrics, geometries, and retrieval augmented generation text documents to support semantic similarity matching operations. The ChromaDB Vector Store 713 may be hosted on EC2 instances that provide dedicated computing resources for vector database operations and enable efficient storage and retrieval of high-dimensional embedding data. In some cases, the ChromaDB Vector Store 713 may store embeddings for over 600 available health metrics across multiple countries, with each embedding representing semantic relationships and contextual information that facilitate accurate query matching and metric selection processes. The vector store may also maintain embeddings for geospatial data layers, administrative boundary information, and supplementary text documents that provide contextual information for response generation through maximal marginal relevance semantic similarity matching algorithms. The ChromaDB Vector Store 713 may utilize specialized indexing and search algorithms that enable rapid similarity comparisons between user query embeddings and stored metric embeddings to identify the most relevant health indicators for analysis.

The second component group within the AWS Cloud Infrastructure 700 may comprise Cloud Data Pipeline Components 720 that orchestrate automated data processing workflows and computational operations when requested datasets are not immediately available in storage systems. The Cloud Data Pipeline Components 720 may include an HSR Event Bus 721 that receives events from the Fine-Tuned LLMs 711 and coordinates the initiation of data processing pipelines based on query requirements and data availability assessments. In some cases, the HSR Event Bus 721 may utilize AWS EventBridge services to manage event routing and trigger appropriate processing workflows when specific health metrics or geographic datasets need to be generated or updated. The event bus architecture may enable asynchronous processing operations that allow the system to handle complex analytical requests without blocking user interface responsiveness or interfering with concurrent query processing activities. The HSR Event Bus 721 may also incorporate event filtering and routing logic that directs different types of processing requests to appropriate computational resources based on the complexity and resource requirements of the analytical operations.

With continued reference to FIG. 5, the Cloud Data Pipeline Components 720 may also include Lambda Functions 722 that execute automated data processing workflows triggered by events from the HSR Event Bus 721 and perform computational operations such as data synthesis, statistical modeling, and geographic aggregation. The Lambda Functions 722 may provide serverless computing capabilities that automatically scale processing resources based on demand and enable efficient execution of data analysis pipelines without maintaining dedicated server infrastructure. In some cases, the Lambda Functions 722 may include 20-minute processing time estimates that inform users about expected completion timeframes for complex analytical operations that generate health metric datasets or perform spatial analysis calculations. The lambda-based architecture may also enable parallel processing of multiple data requests and provide cost-effective computational resources that are allocated dynamically based on processing requirements. The Cloud Data Pipeline Components 720 may further include a Container Registry 723 that stores Docker containers containing the code definitions for each lambda function and provides version control and deployment management capabilities for the automated processing workflows.

The third component group within the AWS Cloud Infrastructure 700 may encompass an S3 Data Lake 730 that provides distributed storage services for health datasets, metadata information, and analytical results generated by the orchestration system. The S3 Data Lake 730 may include Metric Datasets 731 that contain structured health indicator information organized by geographic levels, temporal periods, and metric categories to support diverse analytical requirements and query processing operations. In some cases, the Metric Datasets 731 may encompass multiple years of health data stored in various file formats and organizational structures that require parameter-specific access protocols to ensure accurate data retrieval and processing. The data lake architecture may provide scalable storage capabilities that can accommodate large-scale health datasets while maintaining data accessibility and retrieval performance for concurrent user queries. The S3 Data Lake 730 may also include Metadata Dictionaries 732 that contain comprehensive information about data file locations, naming conventions, temporal coverage, geographic scope, and additional parameters that govern data access and retrieval operations for the stored health metrics and geospatial datasets.

As further shown in FIG. 5, the S3 Data Lake 730 may also incorporate Export Artifacts 733 that provide temporary storage for query results, visualizations, and analytical outputs that users can download and access beyond the immediate interface display capabilities. The Export Artifacts 733 may utilize temporary S3 buckets that generate download links for users to access detailed datasets, high-resolution visualizations, and comprehensive reports in multiple file formats including spreadsheet documents, image files, geographic data formats, and structured analytical reports. In some cases, the export functionality may provide time-limited access to generated results while maintaining data security and storage efficiency through automated cleanup processes that remove temporary files after specified retention periods. The Export Artifacts 733 may also include metadata information about the analytical operations performed, data sources utilized, and geographic scope of the results to provide users with comprehensive documentation of the query processing and analysis methods applied to generate the exported information.

The fourth component group within the AWS Cloud Infrastructure 700 may comprise Web Deployment 740 components that provide user interface access and authentication services for the orchestration system through cloud-based web hosting and security mechanisms. The Web Deployment 740 may include an Web Server 741 that hosts the user interface application and coordinates communication between user interactions and the underlying data processing infrastructure components. In some cases, the Web Server 741 may be deployed as a web application on AWS EC2 virtual machines that provide dedicated computing resources for user interface operations and ensure responsive performance during concurrent user sessions. The web server deployment may also incorporate load balancing and auto-scaling capabilities that adjust computing resources based on user demand and maintain system availability during peak usage periods or high-complexity query processing activities.

With continued reference to FIG. 5, the Web Deployment 740 may also include a Docker Container 742 that contains the Web Server 741 application code and provides containerization for ease of deployment and version control across different computing environments. The Docker Container 742 may enable consistent deployment procedures and facilitate system updates, maintenance operations, and scaling activities without disrupting user access or data processing capabilities. In some cases, the containerized deployment approach may also support development and testing workflows that enable rapid iteration and improvement of the user interface and orchestration system functionality. The Web Deployment 740 may further include a Route 53 Domain 743 that provides domain name services and web traffic routing capabilities to ensure reliable user access to the orchestration system through standard web browsers and internet connections. The domain services may also incorporate DNS management and traffic distribution features that optimize user connection performance and provide geographic load balancing when appropriate for the user base and system deployment architecture.

The Web Deployment 740 may also incorporate AWS Cognito 744 that provides access control and user authentication services to ensure secure system access and protect sensitive health data from unauthorized users or malicious access attempts. The AWS Cognito 744 may implement multi-factor authentication, user session management, and role-based access control mechanisms that align with healthcare data security requirements and regulatory compliance standards. In some cases, the authentication system may also support integration with organizational identity management systems and enable single sign-on capabilities for institutional users such as public health departments, healthcare organizations, and emergency management agencies. The Web Deployment 740 may also explore Django-based alternatives for authentication mechanisms that provide additional customization options and integration capabilities with the Python-based orchestration system components and data processing workflows.

The interconnections between the component groups within the AWS Cloud Infrastructure 700 may enable comprehensive data flow and processing coordination that supports the orchestration system functionality from query reception through response generation and delivery. The Fine-Tuned LLMs 711 may communicate with the HSR Event Bus 721 to trigger data processing pipelines when requested health metrics or geospatial datasets are not immediately available in the S3 Data Lake 730, enabling automated generation of analytical results through the Lambda Functions 722 that access the Container Registry 723 for processing code and store results back to the Metric Datasets 731 and Export Artifacts 733. In some cases, the ChromaDB Vector Store 713 may provide embedding-based similarity matching services that support metric selection and geographic parameter extraction operations performed by the Fine-Tuned LLMs 711, while the DynamoDB Chat History 712 maintains conversation context that enables coherent multi-turn interactions and query refinement processes. The Web Server 741 may coordinate user interface operations with all infrastructure components through the Docker Container 742 deployment while utilizing the Route 53 Domain 743 and AWS Cognito 744 services to provide secure and accessible web-based access to the orchestration system capabilities and analytical results.

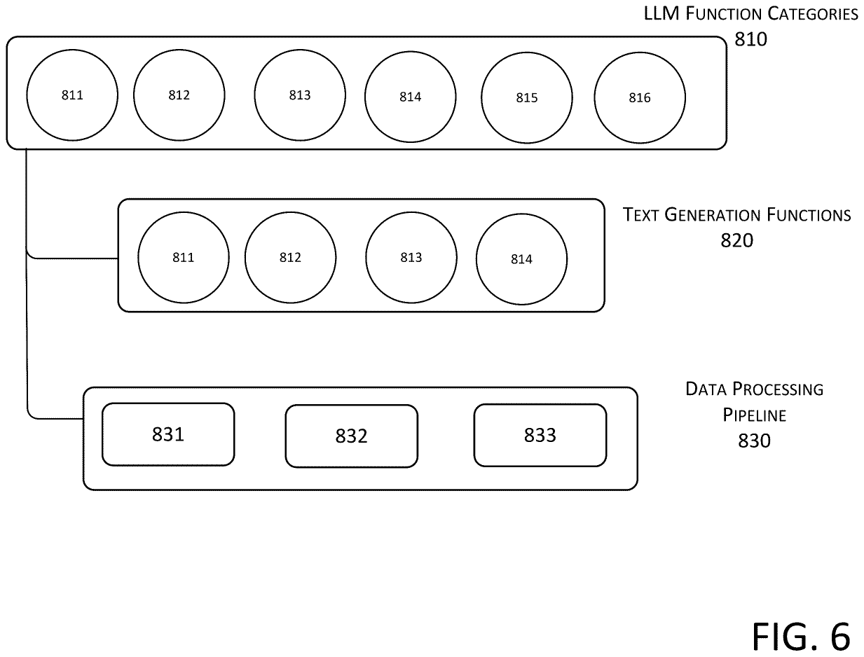

Referring to FIG. 6, an Orchestration System 800 may be implemented to coordinate multiple large language model functions and data processing operations that enable comprehensive query interpretation and response generation for health data analysis applications. The Orchestration System 800 may comprise two main categories of LLM functions that work together to process natural language queries and orchestrate data retrieval operations through specialized computational workflows. In some cases, the Orchestration System 800 may utilize dedicated fine-tuned language models that are designed for specific tasks rather than general-purpose models, with each custom prompt becoming a dedicated fine-tuned LLM that provides enhanced accuracy and performance for particular orchestration functions. The system architecture may enable coordinated processing of user queries through multiple specialized components that handle different aspects of query interpretation, parameter extraction, and data orchestration activities. The Orchestration System 800 may also incorporate data processing pipeline components that manage the flow of information between different LLM functions and coordinate the execution of analytical operations based on extracted query parameters and identified data requirements.

The first category within the Orchestration System 800 may include LLM Function Categories 810 that encompass picker functions designed to select the most relevant options from predefined lists based on user query content and semantic similarity matching algorithms. The picker functions may utilize natural language comprehension capabilities to analyze query text and identify appropriate selections from available options such as geographic areas, health metrics, administrative boundaries, and geospatial features. In some cases, the picker functions may function as custom python functions that take two inputs including the user query and the context of options to select from, which are passed to the LLM from python processing routines. The picker functions may generally produce two possible outputs including the selected relevant option or a null response of “empty_string” when no suitable match is found within the available options. The integration of LLMs within the broader programmatic framework may allow for increased programmatic-based orchestration flexibility and expand the capabilities of the generative AI front-end without requiring the LLM itself to perform complex calculations or analyses, as those tasks may be off-loaded to tools better suited for computational operations.

As further shown in FIG. 6, the LLM Function Categories 810 may include a selectMetricExtent Function 811 that identifies and returns the geographical area from a provided list that may be most relevant to or explicitly mentioned in the user query. The selectMetricExtent Function 811 may analyze user queries to identify geographical references and match such references against available country options within the metric database to ensure accurate country selection based on query content. In some cases, the selectMetricExtent Function 811 may be trained to recognize various forms of country names, abbreviations, and geographic identifiers including formal country designations, common geographic abbreviations, and contextual spatial references that users may employ when specifying areas of interest. The function may utilize natural language comprehension capabilities to parse geographic context from user queries and return the most appropriate country designation for subsequent data filtering operations, while also handling cases where multiple countries are referenced or where geographic scope may be ambiguous within the query text. The selectMetricExtent Function 811 may return “empty_string” responses when no geographical area from the provided list matches the query content, enabling appropriate error handling and alternative processing pathways within the orchestration workflow.

The LLM Function Categories 810 may also incorporate a selectRelevantMetric Function 812 that identifies and returns the health metric from a provided list that may be most relevant to or explicitly mentioned in the user query content. The selectRelevantMetric Function 812 may analyze filtered metric options against user query content to identify the most appropriate health indicator or dataset for addressing query requirements through semantic similarity matching algorithms that compare query embeddings with metric embeddings. In some cases, the selectRelevantMetric Function 812 may consider contextual factors such as the type of analysis requested, the geographic scale specified, and the temporal scope mentioned in the query to ensure that the selected metric aligns with user intentions and analytical requirements. The function may be trained to recognize various forms of health metric references including formal indicator names, common health terminology, and colloquial descriptions of health conditions or risk factors that users may employ when specifying analytical interests. The selectRelevantMetric Function 812 may process lists that contain health indicators such as Hurricane Health Risk Index, social demographic factors, healthcare access measurements, and population health statistics to identify the most appropriate metric for query processing operations.

With continued reference to FIG. 6, the LLM Function Categories 810 may further include a selectGeomLevel Function 813 that identifies and returns the geographic level from a provided list that may be most relevant to or explicitly mentioned in the user query. The selectGeomLevel Function 813 may analyze user queries to determine appropriate spatial resolution for analysis based on geographic scale specifications such as state, county, ZIP code, census tract, or other administrative boundary levels mentioned in the query text. In some cases, the selectGeomLevel Function 813 may be trained to recognize various forms of geographic level references including formal administrative designations, common geographic terminology, and contextual spatial scale indicators that users may employ when specifying analytical scope preferences. The function may utilize natural language comprehension capabilities to parse spatial scale context from query text and return appropriate geographic level specifications that align with available data structures and analytical capabilities within the health metric datasets. The selectGeomLevel Function 813 may handle cases where multiple geographic levels are mentioned or where spatial scale may be hierarchically nested within query specifications, enabling accurate parameter extraction for subsequent data retrieval and filtering operations.

The LLM Function Categories 810 may also encompass a checkAdditionalParams Function 814 that identifies and returns additional parameter values from provided lists that may be most relevant to or explicitly mentioned in user queries. The checkAdditionalParams Function 814 may analyze query content to identify specific parameter specifications that govern data access and retrieval operations for health metrics that require additional categorical or classification parameters beyond geographic and temporal specifications. In some cases, the checkAdditionalParams Function 814 may process parameter lists that include healthcare facility types such as hospitals, women's health clinics, or mental health facilities when users request distance-to-care analyses or healthcare access measurements. The function may be trained to recognize various forms of parameter references including formal category names, common terminology, and contextual parameter indicators that users may employ when specifying analytical requirements. The checkAdditionalParams Function 814 may return “empty_string” responses when no additional parameter values match the query content, enabling the system to apply default parameter values specified in metadata dictionaries for the selected health metrics.

As further shown in FIG. 6, the LLM Function Categories 810 may include a selectRelevantGeometry Function 815 that identifies and returns geospatial features from provided lists that may be most relevant to or explicitly mentioned in user queries. The selectRelevantGeometry Function 815 may analyze user query content to identify specific geographic features, boundary names, or spatial entities that are explicitly mentioned within query text and match such references against available geometry names within geospatial datasets. In some cases, the selectRelevantGeometry Function 815 may be trained to recognize various forms of geometric nomenclature including official boundary names, alternative geographic designations, and contextual spatial references such as hurricane impact areas, drought severity zones, or healthcare facility locations. The function may utilize semantic similarity matching algorithms to compare query content with geometry name embeddings and identify the most appropriate spatial features for analysis operations. The selectRelevantGeometry Function 815 may handle cases where multiple geometry names are referenced or where spatial scope may be ambiguous within query specifications, enabling accurate selection of geospatial components that support map visualization and spatial analysis activities.

The LLM Function Categories 810 may further incorporate a selectAdminFilter Function 816 that identifies and returns administrative boundary levels from provided lists that may be most relevant to or explicitly mentioned in user queries for data filtering operations. The selectAdminFilter Function 816 may analyze user query content to identify which administrative boundary levels are referenced or implied within query text and match such references against available administrative columns within health metric datasets. In some cases, the selectAdminFilter Function 816 may be trained to recognize various forms of geographic references including formal administrative names, common abbreviations, and colloquial geographic identifiers that users may employ when specifying areas of interest for analysis. The function may utilize natural language comprehension capabilities to parse geographic context and determine which administrative levels provide the most appropriate filtering scope for user queries, while also handling cases where multiple administrative levels are mentioned or where geographic scope may be hierarchically nested within query specifications. The selectAdminFilter Function 816 may focus on identifying administrative levels that match the region of interest in queries rather than the geographic level for data presentation, enabling accurate filtering parameter extraction for subsequent data processing operations.

With continued reference to FIG. 6, the LLM Function Categories 810 may also include a selectAdminFilterValue Function 817 that identifies and returns specific administrative names from provided lists that may be most relevant to or explicitly mentioned in user queries. The selectAdminFilterValue Function 817 may analyze query content to identify particular geographic areas, administrative boundaries, or jurisdictional entities that users specify as areas of interest for health data analysis operations. In some cases, the selectAdminFilterValue Function 817 may process lists of administrative names such as state names, county designations, city names, or other geographic identifiers to determine which specific areas should be included in data filtering operations. The function may be trained to recognize various forms of administrative name references including formal jurisdictional names, common geographic abbreviations, and alternative designations that users may employ when specifying geographic areas of interest. The selectAdminFilterValue Function 817 may utilize natural language comprehension capabilities to parse specific geographic identifiers from query text and return appropriate administrative name specifications that enable accurate data filtering based on user-specified geographic scope requirements.

The LLM Function Categories 810 may further encompass a selectGeomName Function 818 that identifies and returns specific geometry names from provided lists that may be explicitly mentioned in user queries for geospatial analysis operations. The selectGeomName Function 818 may analyze user query content to identify particular geographic features, spatial entities, or named geospatial elements that users reference when requesting analysis of specific geographic phenomena or spatial datasets. In some cases, the selectGeomName Function 818 may process lists of geometry names such as hurricane identifiers, drought severity classifications, healthcare facility names, or other spatial feature designations to determine which specific geospatial elements should be included in analysis operations. The function may be trained to recognize explicit mentions of geometry names within query text while distinguishing between general category requests and specific named entity references that require precise geospatial feature selection. The selectGeomName Function 818 may return “empty_string” responses when no specific geometry names are explicitly mentioned in queries, enabling the system to proceed with general category-based geospatial analysis rather than specific named feature analysis.

As further shown in FIG. 6, the Orchestration System 800 may also include Text Generation Functions 820 that utilize LLMs for more traditional natural language generation activities within the orchestration workflow, including parsing input user queries and generating final responses to users based on analytical results and data context information. The Text Generation Functions 820 may complement the picker functions by providing natural language processing capabilities that handle query modification, response generation, and numerical parameter extraction operations that require text analysis and generation rather than selection from predefined option lists. In some cases, the Text Generation Functions 820 may utilize advanced natural language generation capabilities to transform statistical information, geographic data, and analytical results into accessible explanations that can be understood by users without technical backgrounds in data analysis or statistics. The text generation capabilities may also incorporate contextual information about data sources, analytical methods, and geographic scope to provide users with comprehensive understanding of analysis basis and limitations while maintaining scientific accuracy through clear and engaging language that facilitates effective communication of health data insights.

The Text Generation Functions 820 may include an updateUserQuery Function 821 that takes chat history and original user queries as inputs and generates updated user queries that incorporate contextual information from previous interactions. The updateUserQuery Function 821 may analyze conversation history to identify relevant context, parameter specifications, or geographic references from previous queries that may inform the interpretation and processing of new user queries. In some cases, the updateUserQuery Function 821 may modify query content to reflect information from new queries while retaining the same structure and context as original queries, such as replacing geographic references, temporal specifications, or metric categories based on updated user requirements. The function may utilize natural language comprehension and generation capabilities to create coherent query modifications that preserve user intent while incorporating relevant contextual information from chat history records. The updateUserQuery Function 821 may generate updated queries that maintain consistency with previous interaction patterns while accommodating new analytical requirements or parameter specifications that users provide in subsequent queries.

With continued reference to FIG. 6, the Text Generation Functions 820 may also incorporate a determineFinalQuery Function 822 that takes original queries and updated queries as inputs and determines which query version should be utilized for subsequent orchestration processing operations. The determineFinalQuery Function 822 may analyze both query versions to assess which option provides more complete information, clearer analytical requirements, or better alignment with available data and processing capabilities within the orchestration system. In some cases, the determineFinalQuery Function 822 may prioritize original queries when such queries contain sufficient information and clear analytical specifications, while selecting updated queries when the original queries lack context or specificity that can be provided through chat history integration. The function may return either “use_original” or “use_updated” responses based on the LLM assessment of query completeness, clarity, and analytical feasibility. The determineFinalQuery Function 822 may utilize natural language comprehension capabilities to evaluate query quality and determine which version provides the most appropriate foundation for subsequent metric selection, parameter extraction, and data orchestration operations.

The Text Generation Functions 820 may further include an extractTopBottomNum Function 823 that analyzes user queries to identify numerical specifications that indicate how many results users wish to receive in responses to their analytical requests. The extractTopBottomNum Function 823 may process query text to identify requests for specific numbers of results such as top ten areas with highest risk values, five counties with lowest healthcare access scores, or other numerical result specifications that users may include in their queries. In some cases, the extractTopBottomNum Function 823 may be trained to recognize various forms of numerical requests including explicit numbers, ordinal references, and contextual quantity indicators such as “most,” “least,” “top,” or “bottom” that users may employ when specifying result scope preferences. The function may extract requested numbers between 1 and 50 based on query content analysis, while returning “empty_string” responses when no specific result quantities are mentioned in queries. The extractTopBottomNum Function 823 may enable the system to apply appropriate default values that provide comprehensive yet manageable response sizes when users do not specify particular result count preferences.

As further shown in FIG. 6, the Text Generation Functions 820 may also encompass a finalResponse Function 824 that generates direct and concise answers for user queries based solely on data context strings that include relevant retrieval augmented generation information, highest and lowest values, and descriptive statistics of selected metrics from query processing operations. The finalResponse Function 824 may utilize advanced natural language generation capabilities to transform statistical information, geographic data, and analytical results into coherent explanations that maintain scientific accuracy while using accessible language that facilitates effective communication of health data insights. In some cases, the finalResponse Function 824 may be trained to generate responses that incorporate contextual information about data sources, analytical methods, and geographic scope to provide users with comprehensive understanding of analysis basis and limitations. The function may handle various response formats including detailed explanations, summary statements, and action-oriented recommendations that align with different user needs and decision-making contexts. The finalResponse Function 824 may process data context strings that combine calculated summary statistics, extracted top and bottom values, geographic identifiers, and relevant metadata to create structured information for natural language response generation.

With continued reference to FIG. 6, the Orchestration System 800 may also include a Data Processing Pipeline 830 that coordinates the execution of query processing operations and manages the flow of information between different LLM functions and data orchestration components. The Data Processing Pipeline 830 may provide systematic coordination of query interpretation, parameter extraction, and data orchestration activities through specialized processing modules that handle different aspects of the orchestration workflow. In some cases, the Data Processing Pipeline 830 may enable sequential processing of user queries through multiple stages that progressively refine query understanding, extract relevant parameters, and coordinate data retrieval and analysis operations based on LLM function outputs. The pipeline architecture may facilitate coordinated processing that ensures appropriate information flow between picker functions, text generation functions, and data processing operations while maintaining consistency and accuracy throughout the orchestration workflow. The Data Processing Pipeline 830 may also incorporate error handling and validation mechanisms that ensure robust processing of diverse query types and graceful handling of processing limitations or data availability constraints.

The Data Processing Pipeline 830 may include a Query Parser 831 that processes incoming user queries and coordinates the initial interpretation and analysis operations that determine query intent and extract preliminary parameter information. The Query Parser 831 may interface with the Text Generation Functions 820 to handle chat history integration, query updating, and final query determination operations that establish the foundation for subsequent processing activities. In some cases, the Query Parser 831 may coordinate with vector embedding creation processes that transform natural language queries into numerical representations suitable for semantic similarity matching operations with available health metrics and geospatial datasets. The Query Parser 831 may also manage the initial stages of query processing that determine geographic scope, temporal parameters, and analytical requirements based on natural language content analysis. The parser functionality may enable the system to handle diverse query formats and user interaction patterns while maintaining consistent processing workflows that support accurate parameter extraction and data orchestration operations.

As further shown in FIG. 6, the Data Processing Pipeline 830 may also incorporate a Parameter Extractor 832 that coordinates the execution of picker functions and manages the extraction of specific parameters needed for data retrieval and processing operations. The Parameter Extractor 832 may interface with the LLM Function Categories 810 to execute metric selection, geographic parameter extraction, administrative boundary identification, and geospatial feature selection operations based on query content analysis and semantic similarity matching results. In some cases, the Parameter Extractor 832 may coordinate multiple picker function executions in sequence to build comprehensive parameter sets that include country specifications, metric selections, geographic levels, additional parameters, geometry selections, and filtering criteria needed for data access and processing operations. The Parameter Extractor 832 may also manage validation and error handling processes that ensure extracted parameters align with available data structures and processing capabilities within the orchestration system. The parameter extraction coordination may enable systematic processing of complex queries that require multiple parameter specifications while maintaining accuracy and consistency across different extraction operations.

The Data Processing Pipeline 830 may further include a Data Orchestrator 833 that coordinates data retrieval, filtering, and analysis operations based on parameters extracted through the Query Parser 831 and Parameter Extractor 832 processing activities. The Data Orchestrator 833 may manage access to the S3 Data Lake 730 components including Metric Datasets 731 and Metadata Dictionaries 732 to retrieve health data and geospatial information that corresponds to extracted parameters and query requirements. In some cases, the Data Orchestrator 833 may coordinate with the Cloud Data Pipeline Components 720 to initiate Lambda Functions 722 when requested datasets are not immediately available in storage systems, enabling automated generation of analytical results through computational workflows. The Data Orchestrator 833 may also manage data filtering operations that apply geographic constraints, administrative boundary limitations, and geospatial intersection calculations based on extracted parameters and selected geospatial features. The orchestration coordination may enable comprehensive data processing workflows that transform raw health datasets into filtered, analyzed, and formatted information suitable for visualization generation and natural language response creation through the finalResponse Function 824 and associated output generation processes.

The orchestration system may integrate multiple processing components through coordinated data flow pathways that enable comprehensive health data analysis from initial user query reception through final output generation and delivery. The system integration may encompass natural language processing operations, data retrieval mechanisms, geospatial analysis capabilities, and visualization generation processes that work together to transform user queries into actionable health insights. In some cases, the integrated system architecture may coordinate the execution of fine-tuned language model functions with cloud-based data processing pipelines to ensure that user queries receive appropriate analytical treatment regardless of data availability or computational complexity requirements. The system integration may also incorporate real-time coordination between vector embedding operations, semantic similarity matching algorithms, and data orchestration processes that enable accurate query interpretation and parameter extraction across diverse health data domains. The comprehensive integration approach may facilitate seamless transitions between different processing stages while maintaining data consistency and analytical accuracy throughout the orchestration workflow.

The data flow within the integrated system may begin with user query reception through conversational interfaces and proceed through vector embedding creation processes that transform natural language content into numerical representations suitable for computational analysis. The embedded query representations may then flow through semantic similarity matching operations that compare user queries against comprehensive databases of health metric embeddings to identify relevant analytical targets and data sources. In some cases, the data flow may incorporate chat history integration mechanisms that combine current query content with previous interaction context to create enhanced query representations that reflect ongoing analytical conversations and parameter refinements. The enhanced query processing may then trigger coordinated execution of multiple fine-tuned language model functions that extract geographic parameters, select appropriate health metrics, and identify relevant geospatial features based on query content analysis and contextual understanding.

Following parameter extraction operations, the integrated system may coordinate data retrieval processes that access distributed cloud storage systems to obtain health datasets and geospatial information corresponding to extracted query parameters. The data retrieval coordination may involve metadata dictionary consultations that provide file location information, parameter specifications, and data structure details needed to access appropriate datasets from the cloud data lake architecture. In some cases, the system integration may incorporate automated pipeline initiation mechanisms that trigger computational workflows when requested datasets are not immediately available in storage systems, enabling dynamic generation of analytical results through serverless computing functions. The pipeline coordination may also include processing time estimation capabilities that inform users about expected completion timeframes for complex analytical operations while maintaining system responsiveness for concurrent query processing activities.

The retrieved health data and geospatial information may flow through coordinated filtering operations that apply geographic constraints, administrative boundary limitations, and spatial intersection calculations based on extracted query parameters and user specifications. The filtering coordination may integrate administrative boundary selection processes with geospatial feature identification operations to create comprehensive data subsets that align with user analytical requirements and geographic scope preferences. In some cases, the integrated filtering approach may incorporate multiple hierarchical geographic levels simultaneously to create nested spatial constraints that reflect complex geographic relationships within health data structures. The filtered data may then proceed through statistical analysis operations that generate summary measures, identify extreme values, and calculate distributional characteristics that provide quantitative foundations for subsequent response generation activities.

The system integration may culminate in coordinated output generation processes that create multiple presentation formats including natural language responses, geospatial visualizations, statistical tables, and various chart types that accommodate diverse user communication needs and analytical preferences. The output coordination may integrate natural language generation capabilities with visualization creation processes to ensure that textual explanations align with graphical presentations and provide coherent analytical narratives. In some cases, the integrated output generation may also incorporate data export functionality that creates downloadable datasets, high-resolution visualizations, and comprehensive reports that extend analytical capabilities beyond immediate interface display limitations. The export coordination may utilize temporary cloud storage mechanisms that provide time-limited access to generated results while maintaining data security and storage efficiency through automated cleanup processes.

The system may incorporate a Geospatial Exploratory Platform that combines interactive dashboard capabilities with language model orchestrator functionality to provide enhanced data visualization and analytical interaction capabilities. The Geospatial Exploratory Platform may enable users to select data layers and visualization components through drag-and-drop interface mechanisms while simultaneously utilizing natural language query processing to populate dashboard elements automatically based on user requests. In some cases, the platform integration may allow users to request specific analytical content through conversational interfaces, such as asking the system to populate dashboards with distance-to-care measurements and chronic health condition prevalence data, which may trigger automated metric identification and dashboard configuration processes. The platform may also enable users to apply data filters and modify visualization parameters through natural language commands that are processed by the orchestrator components and translated into appropriate dashboard modifications and data constraint applications.

The interactive dashboard capabilities within the Geospatial Exploratory Platform may provide users with direct manipulation tools for selecting geographic layers, health metrics, and visualization formats while maintaining integration with the language model orchestrator functions that enable conversational interaction with displayed data. Users may interact with dashboard components through traditional interface elements such as dropdown menus, checkbox selections, and slider controls while also utilizing natural language queries to request specific analytical operations or data filtering activities. In some cases, the platform may enable users to ask questions about data currently displayed within the dashboard, triggering natural language response generation processes that provide explanations and insights based on the visible analytical content. The conversational dashboard interaction may also include automatic map panning and zoom operations that focus visualization displays on geographic areas referenced in user queries, providing coordinated visual and analytical responses to natural language requests.

The dashboard integration may also incorporate real-time data filtering capabilities that respond to natural language commands by automatically applying geographic constraints, temporal limitations, or categorical filters to displayed datasets without requiring manual interface manipulation. The language model orchestrator components may interpret user filtering requests and translate such requests into appropriate data processing operations that modify dashboard content dynamically based on conversational input. In some cases, the integrated platform may enable users to request comparative analyses or multi-metric visualizations through natural language queries that trigger automatic addition of relevant data layers and analytical components to existing dashboard configurations. The conversational dashboard control may provide users with flexible analytical exploration capabilities that combine the precision of direct interface manipulation with the accessibility and efficiency of natural language interaction for complex analytical operations.

The system may incorporate advanced analytical capabilities through data processing pipelines that execute predictive modeling operations including synthetic population modeling and future prediction analyses that extend beyond traditional descriptive statistical approaches. The synthetic population modeling capabilities may generate hypothetical demographic and health characteristic distributions that enable “what if” analytical scenarios for policy planning, resource allocation, and intervention impact assessment activities. In some cases, the predictive analytics integration may utilize machine learning algorithms and statistical modeling techniques to project future health trends, demographic changes, and risk factor distributions based on historical data patterns and specified scenario parameters. The advanced analytical capabilities may also incorporate uncertainty quantification mechanisms that provide confidence intervals and sensitivity analyses for predictive results to ensure appropriate interpretation and application of modeling outputs in decision-making contexts.

The synthetic population modeling functionality may enable users to explore hypothetical scenarios such as demographic shifts, policy interventions, or environmental changes that could affect health outcomes and resource requirements within specified geographic areas. The modeling processes may generate synthetic datasets that maintain statistical consistency with observed population characteristics while enabling exploration of alternative demographic compositions, health risk distributions, and healthcare utilization patterns. In some cases, the synthetic population capabilities may support comparative analyses that evaluate potential impacts of different policy options, resource allocation strategies, or intervention approaches through quantitative modeling of population-level health outcomes. The predictive modeling integration may also enable temporal projection analyses that estimate future health data patterns based on historical trends, demographic projections, and specified assumption sets about environmental or policy changes that could influence health outcomes over time.

The future prediction pipeline capabilities may incorporate multiple analytical approaches including time series forecasting, regression modeling, and machine learning algorithms that generate projections for health metrics, demographic characteristics, and resource utilization patterns based on historical data and user-specified parameters. The prediction processes may account for seasonal variations, long-term trends, and cyclical patterns within health data while incorporating external factors such as policy changes, environmental conditions, or demographic shifts that could influence future outcomes. In some cases, the predictive analytics may generate multiple scenario projections that reflect different assumption sets about future conditions, enabling users to explore ranges of potential outcomes and assess robustness of planning decisions across different possible futures. The advanced analytical integration may also provide automated model validation and performance assessment capabilities that evaluate prediction accuracy and provide guidance about appropriate applications and limitations of generated forecasts for decision-making purposes.

A number of implementations have been described. Nevertheless, it will be understood that various modifications may be made without departing from the spirit and scope of the disclosure. Accordingly, other implementations are within the scope of the following claims.

1. A computer-implemented method for processing health data queries, comprising:
receiving a user query through a conversational interface;
creating vector embeddings of the user query;
comparing the vector embeddings against embeddings of available health metrics stored in a vector database to identify relevant health metrics;
extracting geographical parameters from the user query using fine-tuned large language models;
retrieving health metric data from a cloud data lake based on the identified relevant health metrics and geographical parameters;
filtering the retrieved health metric data based on the geographical parameters;
generating summary statistics for the filtered health metric data; and
generating multiple output formats including a natural language response and data visualizations.

2. The method of claim 1, wherein the fine-tuned large language models comprise specialized functions for at least one of selecting metric extent, selecting relevant metrics, selecting geometry levels, checking additional parameters, selecting relevant geometries, selecting administrative filters, selecting administrative filter values, and selecting geometry names.

3. The method of claim 2, wherein the specialized functions utilize semantic similarity matching algorithms to compare query embeddings with available option embeddings.

4. The method of claim 1, further comprising initiating cloud-based data processing pipelines when health metric data is not immediately available in the cloud data lake.

5. The method of claim 4, wherein the cloud-based data processing pipelines include lambda functions that execute automated data processing workflows triggered by events from an event bus.

6. The method of claim 1, further comprising retrieving geospatial data layers corresponding to the identified health metrics from the cloud data lake.

7. The method of claim 6, further comprising performing intersection overlay geospatial operations when the geospatial data comprises polygon geometries.

8. The method of claim 1, wherein the data visualizations comprise maps, tables, pie charts, and bar charts.

9. The method of claim 1, further comprising storing chat history in a database and updating user queries based on previous interactions.

10. The method of claim 1, wherein the natural language response is generated using retrieval augmented generation from curated authoritative sources.

11. An AI orchestration system for health data analysis, comprising:
a conversational interface configured to receive user queries;
a vector database storing embeddings of available health metrics;
fine-tuned large language models configured to extract parameters from user queries, wherein the fine-tuned large language models comprise picker functions that select relevant options from available datasets based on user queries;
a cloud data lake storing health metric datasets and geospatial data;
a data orchestrator configured to retrieve and filter data based on extracted parameters; and
a visualization engine configured to generate multiple output formats including natural language responses and data visualizations.

12. The system of claim 11, wherein the picker functions comprise a function to identify geographical areas, a function to identify health metrics, and a function to identify geographic levels.

13. The system of claim 12, wherein the selectMetricExtent function analyzes user queries to recognize country names, abbreviations, and geographic identifiers for accurate country selection.

14. The system of claim 11, further comprising cloud data pipeline components including an event bus and lambda functions for processing data requests when health metric data is not immediately available.

15. The system of claim 14, wherein the lambda functions execute automated data processing workflows that generate required datasets through computational operations including data synthesis and statistical modeling.

16. A computer-implemented method for orchestrating health data analysis using artificial intelligence, comprising:
receiving a natural language query from a user;
processing the query using a plurality of fine-tuned large language model functions to extract query parameters, wherein the plurality of fine-tuned large language model functions comprise a function to identify geographical areas, a function to identify health metrics, and a function to identify geographic levels;
retrieving health data from a distributed cloud storage system based on the extracted query parameters;
performing geospatial filtering operations on the retrieved health data; and
generating a comprehensive response comprising natural language explanations and geospatial visualizations based on the filtered health data.

17. The method of claim 16, wherein the plurality of fine-tuned large language model functions further comprise a function to identify geospatial features and a function to identify administrative boundary levels for data filtering operations.

18. The method of claim 17, wherein the function to identify geospatial features analyzes user query content to match references against available geometry names within geospatial datasets including hurricane impact areas, drought severity zones, and healthcare facility locations.

19. The method of claim 16, wherein the geospatial filtering operations comprise performing intersection overlay operations when geospatial data comprises polygon geometries to spatially filter health data points that fall within selected geographic boundaries.

20. The method of claim 19, wherein the intersection overlay operations utilize computational geometry algorithms that account for coordinate system transformations and boundary precision to ensure accurate spatial filtering results.
